(19) United States
(12) Patent Application Publication
(10) Pub. No.: US 2013/0184775 A1
(43) Pub. Date: Jul. 18, 2013

(54) LONG TERM VAGAL NERVE STIMULATION FOR THERAPEUTIC AND DIAGNOSTIC TREATMENT

(71) Applicants: Peng-Sheng Chen, Indianapolis, IN (US); Shien-Fong Lin, Indianapolis, IN (US); Lan S. Chen, Indianapolis, IN (US)

(72) Inventors: Peng-Sheng Chen, Indianapolis, IN (US); Shien-Fong Lin, Indianapolis, IN (US); Lan S. Chen, Indianapolis, IN (US)

(73) Assignee: Indiana University Research & Technology Corporation, Indianapolis, IN (US)

(21) Appl. No.: 13/876,999

(22) PCT Filed: Sep. 16, 2011
(86) PCT No.: PCT/US2011/051924

§ 371 Date: Mar. 29, 2013

Related U.S. Application Data
(60) Provisional application No. 61/388,664, filed on Oct. 1, 2010.

(51) Int. Cl. A61N 1/36

(52) U.S. Cl. CPC A61N 1/36114; A61N 1/36053
USPC 607/17; 607/62

(57) ABSTRACT

A method of nerve stimulation produces therapeutic effects in an organ not directly innervated by the electrically stimulated nerve. The method includes identifying an electrical stimulation reaction threshold of at least one organ directly innervated by a parasympathetic nerve of a subject, identifying a reaction threshold of at least one tissue that is not directly innervated by the parasympathetic nerve of the subject after the parasympathetic nerve is electrically stimulated, and electrically stimulating the parasympathetic nerve with an electrical stimulation signal that is below the identified reaction threshold for the at least one directly innervated organ, but above a reaction threshold for the at least one tissue or organ that is not directly innervated by the parasympathetic nerve of the subject.

### CLAIM OF PRIORITY

This application claims priority from U.S. Provisional Application No. 61/388,664, which is entitled “LONG TERM VAGAL NERVE STIMULATION FOR THERAPEUTIC AND DIAGNOSTIC TREATMENT,” and was filed on Oct. 1, 2010.

### GOVERNMENT INTEREST

This invention was made with government support under grant number HL071140 awarded by the National Institutes of Health. The United States government has certain rights in the invention.

### TECHNICAL FIELD

The present disclosure relates generally to a method for electrical stimulation of nerves and specifically to a method of electrically stimulating the vagus nerve for therapeutic treatment and for observing changes in various bodily functions in a subject undergoing vagal nerve stimulation.

### BACKGROUND

In humans and various vertebrates, the autonomic nervous system includes a sympathetic nervous system and a parasympathetic nervous system. Together, the sympathetic and parasympathetic nervous systems regulate many bodily activities including heart rhythm, skeletal muscle contraction, and bowel activity. In some situations, the sympathetic nervous system and parasympathetic nervous system regulate bodily functions in opposition to one another. That is, the response of an organ or a muscle to nervous stimulation applied by one of the sympathetic and parasympathetic nervous systems is counteracted by stimulation of corresponding nerves in the opposing nervous system. For example, in many mammals including humans, stimulation of sympathetic nerves associated with the heart results in an increase in heart rate, while stimulation of corresponding parasympathetic nerves may result in a decrease in heart rate.

The cardiac autonomic nervous system consists of extrinsic and intrinsic components. Intrinsic components include nerves that originate within the heart, and extrinsic components include nerves that originate outside of the heart. The sympathetic innervation comes from the superior cervical ganglia and the cervicothoracic (stellate) ganglia, which, respectively, communicate with the cervical nerves C1-C3, and with the cervical nerves C7-C8 to the thoracic nerves T1-T2. In addition, the thoracic ganglia (as low as at least the 4^{th} thoracic ganglion) also contribute to the sympathetic innervation of the heart. The superior, middle, and inferior cardiac nerves from these ganglia innervate the heart by following the brachiocephalic trunk, common carotid arteries, and subclavian arteries. The thoracic cardiac nerves in the posterior mediastinum follow a more complex course to reach the heart in the middle mediastinum.

Parasympathetic innervation is mediated by the vagus nerves and divided into superior, middle, and inferior branches. Most of the vagal nerve fibers converge at a distinct fat pad between the superior vena cava and the aorta, referred to as the ‘third fat pad,’ en route to the sinus and atrioventricular nodes. These extrinsic cardiac nerves communicate directly to the intrinsic cardiac nerves to regulate normal heart beats, and may trigger the development of abnormal heart beats (heart rhythm disorders).

Heightened sympathetic nerve activation is known to damage the heart and contributes to the development of heart failure. Beta blockers, which reduce the sympathetic activation of the heart, are standard therapies of various kinds of heart diseases, including heart failure and coronary artery diseases. These therapies are known to improve survival in instances where heightened sympathetic nerve activation is contributing to improper heart function.

In addition to blocking the beta receptors in the heart as a treatment for various types of heart disease, studies have shown that reducing the sympathetic nerve outflow from the stellate ganglia can be effective in treating certain heart rhythm disorders and, perhaps, other types of heart disease. For example, surgical resection of the left stellate ganglion and the T2-T4 thoracic ganglia have shown promise in treating patients with congenital long QT syndrome and arrhythmias associated with heart attacks. In addition, thoracic sympathectomy is the treatment of choice for primary hyperhidrosis (excessive sweating) of the hands, axillae, feet, and face.

While resection of sympathetic nerves has shown efficacy in the treatment of certain types of heart disease and some neurological disorders, these surgical procedures are known to cause side effects. Because surgical resection is a permanent procedure that cannot be reversed, any side effects therefrom may be difficult to manage and could cause long term complications. These side effects, coupled with the permanence of the condition produced by resection, have precluded sympathetic nerve resectioning from becoming a widely practiced treatment in hospitals. Thus, it is desirable to develop methods to reduce the sympathetic outflow without permanently damage the sympathetic nerves.

As discussed above, the heart is also innervated by the parasympathetic nervous system through the cardiac branch of the vagus nerve. FIG. 4 and FIG. 5 provide a simplified depiction of some of the organs, muscles, and systems that interact with the vagus nerve. The vagus nerve begins in the cranial cavity as seen in FIG. 4 at 404, and extends throughout much of the thoracic and abdominal cavities including laryngeal muscles 408, cardiac muscles 412, and the stomach 416. The vagus nerve includes various branches with two major branches being the left vagus and right vagus nerve. As depicted in FIG. 5, in most of the dogs, the left vagus 504 stimulates the atrioventricular (AV) node 512 in the heart, while the right vagus 508 stimulates the sinoatrial node 516. However, the left vagus nerve stimulation can also affect the sinus node 516, while the right vagus nerve stimulation may also affect the AV node 512. Various nerves including the vagus nerves are formed from both afferent and efferent nerve fibers. The afferent nerve fibers conduct nervous signals from tissue or organs in the body to the central nervous system. The efferent nerve fibers conduct nervous signals from the central nervous system to organs or tissue throughout the body.

Existing treatment methods are known that apply electrical stimulation to the vagus nerve to produce a response from one or more of the organs, muscles, glands, or bodily systems that are directly innervated by the vagus nerve. These methods apply an electrical current with a selected current, voltage, frequency, and waveform to produce a measurable response in the innervated organ. However, new therapeutic and diagnostic techniques using vagal nerve stimulation to produce a bodily response in organs or tissue not directly innervated by the cardiac branch of the vagal nerve would be appreciated in the art.

### SUMMARY

In one embodiment, a method for generating an electrical signal for nerve stimulation has been developed. The method includes identifying an electrical stimulation reaction threshold of at least one organ directly innervated by a parasympathetic nerve of a subject, identifying a reaction threshold of at least one tissue that is not directly innervated by the parasympathetic nerve of the subject after the parasympathetic nerve is electrically stimulated, and electrically stimulating the parasympathetic nerve with an electrical stimulation signal that is below the identified reaction threshold for the at least one directly innervated organ, but above a reaction threshold for the at least one tissue or organ that is not directly innervated by the parasympathetic nerve of the subject.

In another embodiment, a method for reducing tyrosine hydroxylase production has been developed. The method includes identifying an electrical stimulation reaction threshold of at least one organ directly innervated by a parasympathetic nerve of a subject, identifying a reaction threshold of at least one sympathetic nerve after the parasympathetic nerve is electrically stimulated, and electrically stimulating the at least one parasympathetic nerve for a preselected time to elicit a phenotypic change in the at least one sympathetic nerve.

In another embodiment, a method for therapeutic treatment through autonomic nerve stimulation is achieved. The method includes identifying an electrical stimulation reaction threshold of at least one organ directly innervated by a first autonomic nerve of a subject, adjusting the electrical stimulation to be below the identified reaction threshold, and applying the electrical stimulation to the first autonomic nerve to produce a phenotypic change within one or more cells comprising a second autonomic nerve.

### BRIEF DESCRIPTION OF THE DRAWINGS

FIG. 1 is a block diagram depicting a process of vagal nerve stimulation.

FIG. 2 is a block diagram depicting an alternative process of vagal nerve stimulation.

FIG. 3A is a slide depicting cells that exhibit tyrosine hydroxylase and cells that do not exhibit tyrosine hydroxylase in sympathetic nervous tissue of a control canine subject.

FIG. 3B is a slide depicting cells that exhibit and that do not exhibit tyrosine hydroxylase in sympathetic nervous tissue of a canine subject that has undergone the process of FIG. 1 or FIG. 2.

FIG. 4 is an anatomical diagram showing the anatomically accepted structure of the vagus nerve extending from the cranial cavity to the abdominal cavity in a typical human.

FIG. 5 is an anatomical diagram showing the anatomically accepted structure of the vagus nerve as it relates to a typical human heart.

### DETAILED DESCRIPTION

For a general understanding of the environment for the system and method disclosed herein as well as the details for the system and method, reference is made to the drawings. In the drawings, like reference numerals have been used throughout to designate like elements. As used herein, the term “protein expression” refers to a measurement of a presence and abundance of one or more proteins in a cell of an organism. A positive protein expression indicates that the protein is present within the cell at or above a predetermined concentration level, and a negative expression indicates that the protein is either absent from the cell or present in concentration levels below the predetermined threshold. Some types of proteins are included in enzymes that act as catalysts for the production of various molecules, including neurotransmitters, within cells of an organism.

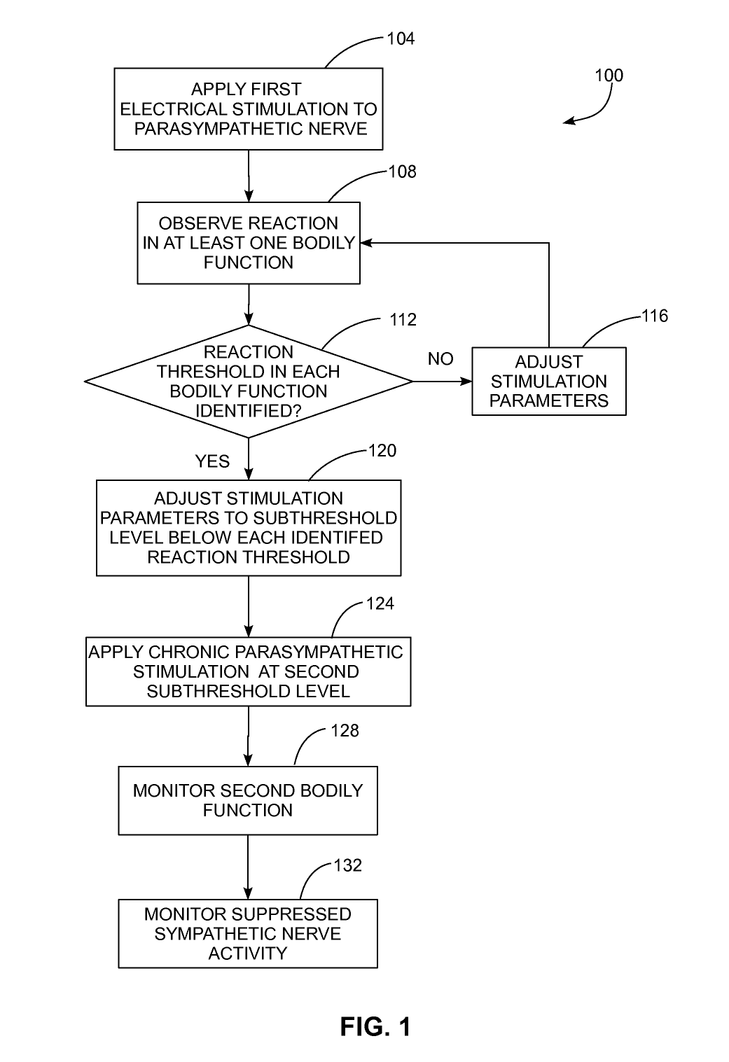

FIG. 1 depicts a process 100 for controlled stimulation of a parasympathetic nerve in a subject. Appropriate subjects for process 100 include any animal having one or more parasympathetic nerve, including human beings, mammals, reptiles, amphibians, birds, fish, and other vertebrates. Process 100 begins by applying a first electrical stimulation to a parasympathetic nerve of a subject (block 104) and observing a reaction to the stimulation (block 108).Various embodiments of process 100 apply the first electrical stimulation to the vagus nerve. According to certain embodiments, one or both of the left and right vagus nerves are electrically stimulated, and the stimulation can be applied to selected nerve fibers within the vagus nerve. In one exemplary embodiment, the first electrical stimulation is applied to afferent nerve fibers in the left vagus nerve. Examples of devices that are suitable for applying various electrical stimulation signals to the vagus nerve include Itrel® brand neurostimulators (available from Medtronic, Inc.) and the NeruoCybernetic Prosthesis System (available from Cyberonics, Inc.).

The first electrical stimulation is typically selected to have an amplitude of current and voltage, frequency, and waveform that produces a measurable response in the function of at least one organ, muscle, or bodily system of the subject. In an exemplary canine subject, an electrical signal having a voltage of 1-20 volts, with 0.25-2 milliamps of current at a frequency in a range of about 10 Hz to about 40 Hz lowered the heart rate of the subject by a measurable amount when applied to the left cervical vagus nerve. In the first iteration, the stimulation is above the threshold for a reaction to the stimulation, indicating that the applied signal exceeds the first reaction threshold (block 112). The term “reaction threshold” as defined herein refers to a level of electrical stimulation that produces a minimally measurable reaction to at least one bodily function in the subject. An alternative “severe reaction threshold” refers to a level of electrical stimulation that produces a medically deleterious reaction in the directly innervated organ of the subject. “Directly innervated” refers to an organ that is in physical contact with a particular branch of an autonomic nerve or nerves and the organ receives stimulus from the autonomic nerve or nerves. Such deleterious reactions may include atrial fibrillation, cardiac arrhythmias, nausea, or loss of muscle control when the vagus nerve is stimulated. Subthreshold stimulation refers to electrical signals that do not produce a measurable reaction to the observed bodily function of the directly innervated organ of the subject. Different subjects may have different reaction thresholds to electrical stimulation, and the reaction threshold may vary over time for a single subject. Depending upon the bodily function under measurement, the same subject may have different reaction thresholds to various electrical stimulation signals.

After an electrical stimulus produces a measured response, process 100 adjusts the parameters of the electrical stimulus to attenuate a magnitude of the response (block 116). Parameters of the electrical stimulus suitable for adjustment include the voltage, current, frequency, phase, and waveform shape of the electrical stimulus signal. The adjustments reduce the magnitude of the reaction to the stimulation exhibited by the subject when the stimulation approached the first reaction threshold. For example, the level of voltage in the applied electrical signal may be lowered in predetermined increments, with additional observations (block 108) of reactions until the first reaction threshold is identified (block 112). The processing of blocks 116, 108, and 112 may iterate until the reaction threshold for at least one bodily function of the innervated organ is identified. In a canine subject, each iteration of the processing of blocks 116, 108, and 112 reduces the measured heart rate of the subject by fewer beats per minute until the applied signal produces no measurable change in the heart rate.

In some configurations, process 100 identifies reaction thresholds for multiple bodily functions to identify an electrical stimulation signal that is below the reaction thresholds for each of the bodily functions. For example, in canine subjects an electrical stimulation signal that is below the reaction threshold for the directly innervated organ (e.g., the heart rate when the vagus nerve is stimulated) may still exceed a reaction threshold for other bodily functions, such as respiration, muscle control, coughing, or drooling, which are associated with organs not directly innervated by the stimulated nerve. Process 100 iterates through blocks 108-116 to identify the reaction threshold for each selected bodily function in the subject. Examples of bodily functions having reaction thresholds identified in process 100 include heart rate, heart arrhythmia such as tachycardia, muscular activity in the gastrointestinal system, perspiration in the extremities such as the palms, drooling, and coughing. The method depicted in these blocks may also be repeated periodically to recalibrate the identified reaction threshold in instances where the reaction threshold varies over time.

After identifying the reaction threshold for the one or more bodily functions, process 100 adjusts the electrical stimulation parameters to a predetermined value below the first identified reaction threshold (block 120). The adjusted electrical stimulation is also referred to as a subthreshold electrical stimulation because the adjusted electrical stimulation parameters are below the identified reaction thresholds for the previously measured bodily functions. In one example, the adjusted stimulation signal has an electrical current with an amperage that is 75% of the electrical current of the identified threshold signal. In another example, the adjusted stimulation signal has a voltage that is one volt less than the voltage of the identified threshold signal. In a canine subject where electrical changes in heart rate are measured during application of the stimulation signals, an exemplary subthreshold electrical signal is a four volt signal with frequency range of 14 Hz to 30 Hz delivering 450 μs electrical pulses. The adjusted subthreshold stimulation may suppress sympathetic nervous activity, including activity observed in the left stellate ganglion and superior left ganglionated plexi. Various other subthreshold signal adjustments are envisioned where the subthreshold signal has a sufficient magnitude to have a therapeutic or diagnostic effect even if the stimulation does not produce a directly measurable reaction.

After identifying the subthreshold electrical signal, the subject receives chronic, or long-term, parasympathetic nerve stimulation applied at the subthreshold level (block 124). The chronic stimulation may include continuous or intermittent application of the subthreshold electrical signal during a treatment period. In one example, process 100 applies electrical stimulation to afferent nerve fibers in the left vagus nerves for a 30 second duration with a one minute gap between stimulation periods. The chronic treatment program may have a duration from several hours, days, weeks, months, or years of treatment. Various other treatment programs, including variable time stimulation periods and treatments that follow the natural diurnal cycles of the subject are also envisioned. Process 100 may be performed iteratively over a period of several weeks or months to identify an optimal level of electrical stimulation for various bodily functions.

Process 100 monitors the activity of a second bodily function in the test patient both during and after the chronic parasympathetic stimulation (block 128). The second bodily function is a bodily function other than the bodily functions used to adjust the stimulation signal described above in blocks 108-120. For example, in one course of treatment process 100 identifies a subthreshold signal that is below a threshold level for producing tachycardia, elevated heart rate, in the subject, and process 100 monitors the severity of coughing in the subject during and after the chronic subthreshold stimulation of block 124. In various embodiments, the second bodily function can include coughing, drooling, perspiration, sympathetic outflow, and other bodily functions that are regulated, at least in part, by the sympathetic nervous system.

The chronic subthreshold stimulation in process 100 provides therapeutic effects to suppress certain undesirable bodily functions in a patient. For example, the stimulation of the parasympathetic nerves can suppress coughing, drooling, muscle spasms, and excessive perspiration in the extremities (hyperhidrosis). As described in more detail below, the low-level parasympathetic nerve stimulation also suppresses activity in the sympathetic nervous system. The suppressed sympathetic nervous activity reduces the effects of the sympathetic outflow that produces stress, elevated blood pressure, and muscle spasms in the body. The effects of the subthreshold stimulation on the second bodily function can last beyond the period of chronic stimulation. Process 100 optionally monitors suppressed levels of sympathetic nervous activity in response to the subthreshold vagal stimulation (block 132). The suppressed activity is detected by measuring the sympathetic nerve activity before the vagal stimulation used to establish the bodily function threshold is applied and then identifying the reduction in the sympathetic nerve activity occurring after the subthreshold stimulation is applied.

In particular, sympathetic nerve activity may be monitored using a wireless monitoring device similar to DSI D70EEE radiotransmitter that enables a subject to remain ambulatory during monitoring. To differentiate the nerve signals from the heart signals (electrocardiogram), the signals should be sampled at or greater than 1,000 times per second. The acquired signals are then high-pass filtered at 150 Hz. The remaining signals having a frequency of at least 150 Hz are then integrated over a series of one minute periods for a total of 24 hours to obtain the baseline sympathetic nerve activities. The same method is used to measure sympathetic nerve activity for 24 hours after the commencement of the vagal nerve stimulation.

The left stellate ganglion nerve activity is an example of measurably suppressed activity following vagal nerve stimulation. The suppression of sympathetic nervous activity has a therapeutic effect for various medical conditions including atrial fibrillation and paroxysmal atrial tachyarrhythmias. Additionally, the subthreshold vagal stimulation reduces side effects in other bodily functions that could interfere with diagnosing medical conditions or with medical treatments. Process 100 minimizes side effects in different bodily functions due to vagal nerve stimulation while continuing to suppress the sympathetic nerve activity. Post-stimulation observations record the long-term effects of a chronic subthreshold vagal stimulation treatment, including measurements of suppressed sympathetic activity after completion of the treatment.

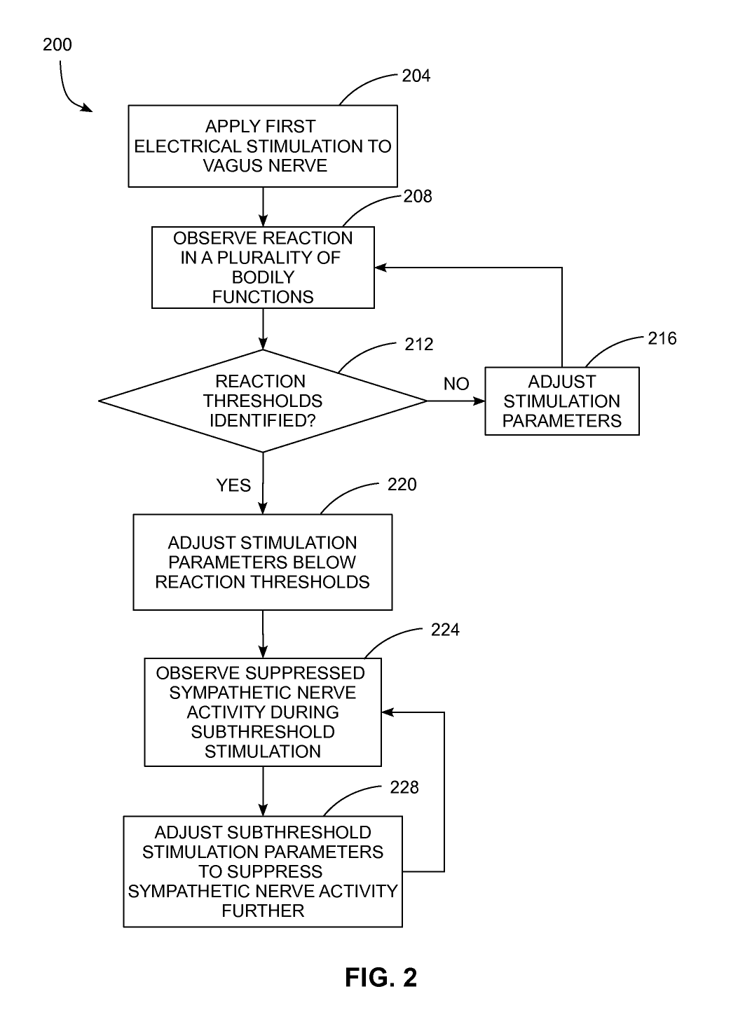

FIG. 2 depicts an alternative process 200 for controlled stimulation of a parasympathetic nerve such as the vagus nerve in a subject to suppress activity in the sympathetic nervous system. Process 200 begins by applying a first electrical stimulation to the vagus nerve of a subject (block 204), observing a reaction in a bodily function (block 208), adjusting parameters of the electrical stimulation (block 216) to identify the reaction threshold for the bodily function (block 212), and then adjusting the electrical stimulation parameters to provide a subthreshold electrical stimulation signal (block 220). The processing in blocks 204-220 may be carried out in a similar manner to the process shown in blocks 104-120 described above.

Process 200 applies the subthreshold electrical stimulation to the vagus nerve while observing suppressed sympathetic nerve activity (block 224). In one embodiment, the subthreshold electrical stimulation is applied to afferent nerve fibers in the left vagus nerve. The vagal nerve stimulation suppresses left stellate ganglion nerve activity (SGNA) and superior left ganglionated plexi nerve activity (SLGPNA), and a wireless monitoring device may measure the activity levels of one or both of these nerves. Process 200 adjusts the electrical stimulation signal to increase the suppression of sympathetic nerve activity (block 228). The electrical stimulation signal remains below the identified threshold level while providing improved suppression of sympathetic nervous activity. The observation of sympathetic nerve activity (block 224) and adjustment of the electrical stimulation signal (block 228) may be iterated to maintain suppression of sympathetic nervous activity for an extended period of time.

In one embodiment of process 200, afferent nerve fibers in the left vagus nerve are stimulated with an identified subthreshold electrical signal to reduce occurrences of atrial fibrillation. This stimulation reaches the heart through the AV node 512 and sinus node 516. The subthreshold stimulation also suppresses sympathetic nervous activity including nervous activity in the left stellate ganglion nerve, even though the sympathetic nervous system is not directly controlled by the vagus nerve. Thus, process 200 provides a method for vagal stimulation that has a therapeutic effect on bodily functions and produces reactions in bodily systems that are not directly innervated by the vagus nerve. As with process 100, process 200 also provides therapeutic benefits to suppress coughing, drooling, perspiration, sympathetic nerve outflow, and other bodily functions that are regulated, at least in part, by the sympathetic nervous system.

Both of processes 100 and 200 may produce structural changes in nervous tissue other than the nervous tissue that receives the electrical stimulation (the “complementary nerves”). For example, processes 100 and 200 both stimulate one autonomic nerve, in this case parasympathetic nerves such as the left and right vagus nerves. The stimulation of the vagus nerves produces changes to the protein expression of complementary autonomic nerves, in this case sympathetic nerves such as the stellate ganglion, even though the sympathetic nerves do not receive any electrical stimulation. In some sympathetic nervous tissue, a reduced percentage of nerve cells express the tyrosine hydroxylase (TH) enzyme after the parasympathetic nerves receive sub-threshold electrical stimulation for a preselected time. TH is an enzyme that catalyzes L-tyrosine into L-DOPA in the presence of tetrahydropbiopterin, and is therefore essential for the production of various neurotransmitters including dopamine, norepinephrine, and epinephrine. When a nerve cell does not express the TH enzyme, the nerve tissue produces lower levels of the corresponding neurotransmitters, and the overall level of activity in the nerve tissue also decreases. As such, according to at least one embodiment, the subthreshold stimulation of a parasympathetic nerve causes a phenotypic change in sympathetic nerve tissue. The phenotypic change in one embodiment is a reduction in the expression of at least one enzyme active in catecholamine production and/or an increase in the expression of at least one enzyme active in acetylcholine production. In another embodiment, the phenotypic change is an upregulation of ion channels in complementary sympathetic nerve tissue. By way of nonlimiting example, the subthreshold stimulation of afferent vagal nerve tissue has been shown to result in upregulation of the SK2 channels in the stellate ganglion. The structural changes produced in the complementary nerves persist for a time after the termination of electrical stimulation to the stimulated nerve. As such, phenotypic changes in complementary nerve cells and tissues are maintained even in the absence of subthreshold stimulation in the electrically stimulated nerve. According to at least one embodiment, the phenotypic changes in complementary nerve cells may be maintained for one or more days, several days, one week or more, or multiple weeks in response to the subthreshold stimulation delivered according to at least one embodiment above.

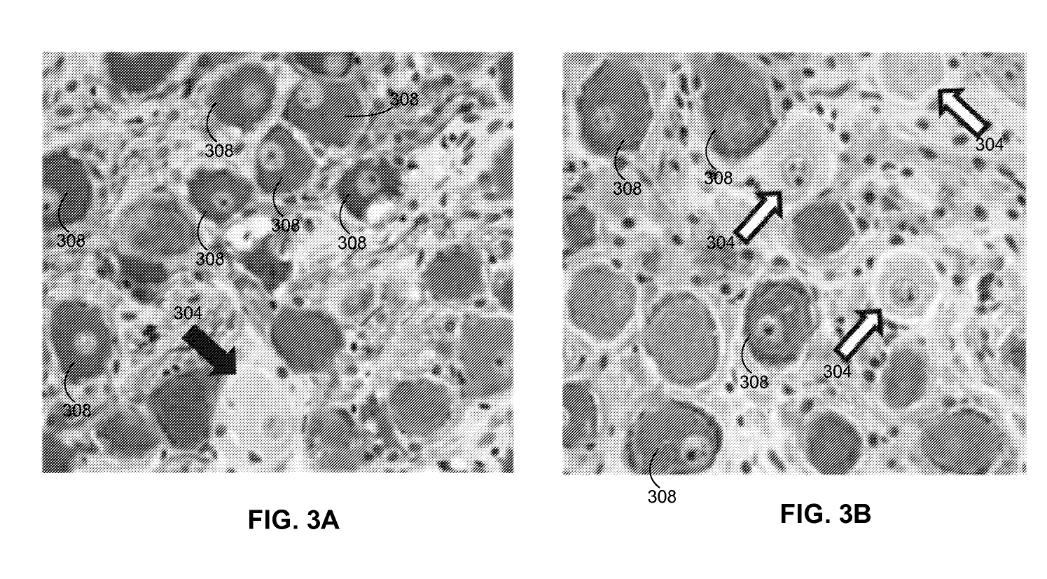

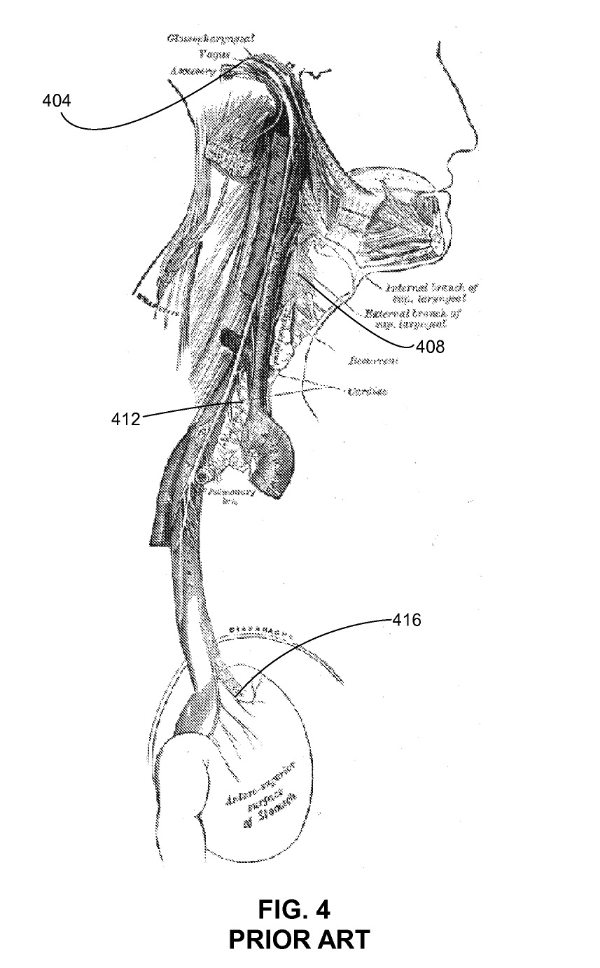

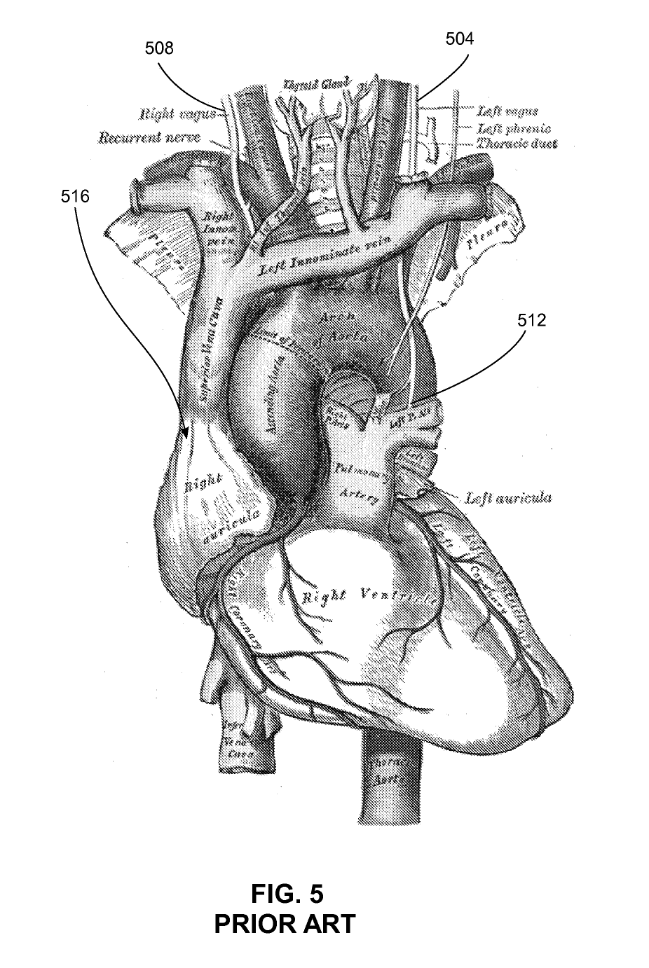

FIG. 3A depicts tissue sample of sympathetic stellate ganglion nerve tissue from a control canine subject that has not undergone the subthreshold vagal nerve stimulation. The cells depicted in FIG. 3A are stained to darken when the cell expresses the TH enzyme, such as cells 308, while lighter color cells such as cell 304 do not express the TH enzyme. Thus, the cells 308 (dark color) are TH positive while the cells 304 (light in color) are TH negative.

FIG. 3B depicts sympathetic stellate ganglion nerve cells in a canine subject that has undergone low-level vagal nerve stimulation as described above in FIG. 1 and FIG. 2. As depicted in FIG. 3B, the proportion of TH negative cells 304 in FIG. 3B is higher than in FIG. 3A. In one embodiment, the percentage of TH negative cells in stellate ganglia of canine subjects that underwent subthreshold vagal stimulation was 11.4% compared to 4.9% in a control group. The cells that do not exhibit tyrosine hydroxylase are more numerous in FIG. 3B than in FIG. 3A, indicating that the process of FIG. 1 or FIG. 2 has significantly altered the expression of tyrosine hydroxylase in the sympathetic nervous tissues.

The TH enzyme is one of a variety of enzymes produced in nervous tissue. Another enzyme, choline acetyltransferase (ChAT) is associated with the production of the neurotransmitter acetylcholine. In the cells depicted above in FIG. 3B, a large proportion (approximately 95%) of the TH negative cells in the sympathetic nervous tissue express the ChAT enzymes after the low-level stimulation. Consequently, the TH negative cells in the sympathetic nerve continue to produce acetylcholine at normal levels.

As depicted above, the structural changes to the protein expression in the stellate ganglion reduce sympathetic nerve outflow. Because there are stimulation-induced structural changes of the nerve structures, there are significant carry-over effects which result in prolonged suppression of the sympathetic outflow even when the vagus stimulation is temporarily deactivated. Therefore, intermittent applications of the subthreshold stimulation on a long-term basis can provide the therapeutic benefits.

While a greater percentage of the cells in the stellate ganglion are TH negative after the parasympathetic stimulation, the suppressed stellate ganglion remains undamaged and continues to function at a reduced activity level. Consequently, the chronic subthreshold vagal stimulation provides therapeutic benefits while avoiding the side effects of existing techniques such as sympathetic nerve resectioning.

While the preferred embodiments have been illustrated and described in detail in the drawings and foregoing description, the same should be considered as illustrative and not restrictive in character. It is understood that only the preferred embodiments have been presented and that all changes, modifications and further applications that come within the spirit of the invention are desired to be protected.

What is claimed is:

1. A method for generating an electrical signal for nerve stimulation, the method comprising:
identifying an electrical stimulation reaction threshold of at least one organ directly innervated by a parasympathetic nerve of a subject;
identifying a reaction threshold of at least one tissue or organ that is not directly innervated by the parasympathetic nerve of the subject after the parasympathetic nerve is electrically stimulated; and
electrically stimulating the parasympathetic nerve with an electrical stimulation signal that is below the identified reaction threshold for the at least one directly innervated organ, but above a reaction threshold for the at least one tissue or organ that is not directly innervated by the parasympathetic nerve of the subject.

2. The method of claim 1 wherein the parasympathetic nerve is a vagus nerve.

3. The method of claim 2 wherein the electrical stimulation is applied to afferent nerve fibers of a left vagus nerve.

4. The method of claim 2 wherein the at least one organ directly innervated by a parasympathetic nerve of a subject is a heart.

5. The method of claim 4 wherein the at least one tissue or organ that is not directly innervated by the parasympathetic nerve of the subject is selected from the group consisting of dermal tissue, skin, intestinal tissue, epithelial tissue, and somatic muscle.

6. The method of claim 1 wherein the reaction threshold of at least one organ directly innervated by a cardiac branch of the parasympathetic nerve of a subject relates to a bodily function selected from increasing or decreasing heart rate.

7. The method of claim 6 wherein the reaction threshold of at least one tissue or organ that is not directly innervated by the cardiac branch of the parasympathetic nerve of the subject relates to a bodily function selected from gastrointestinal activity, perspiration, or somatic muscle activity of the subject.

8. A method for reducing tyrosine hydroxylase production, the method comprising:
identifying an electrical stimulation reaction threshold of at least one organ directly innervated by a parasympathetic nerve of a subject;
identifying a reaction threshold of at least one sympathetic nerve after the parasympathetic nerve is electrically stimulated; and
electrically stimulating the at least one parasympathetic nerve for a preselected time to elicit a phenotypic change in the at least one sympathetic nerve.

9. The method of claim 8 wherein the phenotypic change in the at least one sympathetic nerve is a reduction in the production of at least one enzyme.

10. The method of claim 8 wherein the phenotypic change in the at least one sympathetic nerve is an increase in the production of at least one enzyme.

11. The method of claim 8 or 9 wherein the enzyme is selected from the group consisting of acetyltransferase and tyrosine hydroxylase.

12. The method of claim 8 wherein the phenotypic change in at least one sympathetic nerve is an upregulation of SK2 channels.

13. A method for generating an electrical signal for nerve stimulation, the method comprising:
identifying an electrical stimulation reaction threshold of at least one organ directly innervated by a first autonomic nerve of a subject;
adjusting the electrical stimulation to be below the identified reaction threshold; and
applying the electrical stimulation to the first autonomic nerve to produce a phenotypic change within one or more cells comprising a second autonomic nerve.

14. The method of claim 13 wherein the electrical stimulation is applied to the afferent nerves of the first autonomic nerve.

15. The method of claim 13 wherein the phenotypic change is a lack of expression of one or more enzymes.

16. The method of claim 15 wherein the one or more enzymes are necessary for production of catecholamines or acetylcholine.

17. The method of claim 13 wherein the phenotypic change is maintained after cessation of the electrical stimulation.

18. The method of claim 13 wherein the first autonomic nerve is a vagus nerve, and the second autonomic nerve is a stellate ganglion.

19. The method of claim 13 wherein the first autonomic nerve is a parasympathetic nerve, and the second autonomic nerve is a sympathetic nerve.
